(19) United States
(12) Patent Application Publication
(10) Pub. No.: US 2006/0161217 A1
(43) Pub. Date: Jul. 20, 2006

(54) Methods and systems for treating obesity

(76) Inventors: Kristen N. Jaax, Saugus, CA (US); Todd K. Whitehurst, Santa Clarita, CA (US); Rafael Carbunaru, Studio City, CA (US)

Correspondence Address: STEVEN L. NICHOLS;RADER, FISHMAN & GRAVER PLLC, 10653 S. RIVER FRONT PARKWAY, SUITE 150, SOUTH JORDAN, UT, 84095, US

(21) Appl. No.: 11/315,650

(22) Filed: Dec. 21, 2005

Related U.S. Application Data
(60) Provisional application No. 60/638,609, filed on Dec. 21, 2004.

(51) Int. Cl. A61N 1/08

(52) U.S. Cl. 607/40

(57) ABSTRACT

Methods of treating obesity include applying at least one stimulus to a stimulation site within a patient with an implanted stimulator in accordance with one or more stimulation parameters. Systems for treating obesity include a stimulator configured to apply at least one stimulus to a stimulation site within a patient in accordance with one or more stimulation parameters.

### RELATED APPLICATIONS

The present application claims the priority under 35 U.S.C. §119(e) of previous U.S. Provisional Patent Application No. 60/638,609, filed Dec. 21, 2004, which is incorporated herein by reference in its entirety.

### BACKGROUND

Obesity is one of the most prevalent public heath problems in the United States and affects millions of Americans. An especially severe type of obesity, called morbid obesity, is characterized by a body mass index greater than or equal to 40 or a body weight that is 100 pounds over normal weight.

Recent studies have shown that over 300,000 deaths are caused by obesity in the United States each year. In addition, millions suffer broken bones, social isolation, arthritis, sleep apnea, asphyxiation, heart attacks, diabetes, and other medical conditions that are caused or exacerbated by obesity.

Patients suffering from obesity have very limited treatment options. For example, drugs such as sibutramine, diethylproprion, mazindol, phentermine, phenylpropanolamine, and orlistat are often used to treat obesity. However, these drugs are effective only for short-term use and have many adverse side-effects.

Another treatment option for obesity is surgery. For example, a procedure known as “stomach stapling” reduces the effective size of the stomach and the length of the nutrient-absorbing small intestine to treat obesity. However, surgery is highly invasive and is often associated with both acute and chronic complications including, but not limited to, infection, digestive problems, and deficiency in essential nutrients.

### SUMMARY

Methods of treating obesity include applying at least one stimulus to a stimulation site within a patient with an implanted stimulator in accordance with one or more stimulation parameters.

Systems for treating obesity include a stimulator configured to apply at least one stimulus to a stimulation site within a patient in accordance with one or more stimulation parameters.

### BRIEF DESCRIPTION OF THE DRAWINGS

The accompanying drawings illustrate various embodiments of the present invention and are a part of the specification. The illustrated embodiments are merely examples of the present invention and do not limit the scope of the invention.

FIG. 1A is a diagram of the human nervous system.

FIG. 1B illustrates the autonomic nervous system.

FIG. 2A depicts the lateral surface of the brain.

FIG. 2B depicts, in perspective view, the thalamus and various structures of the brain that make up the limbic system.

FIG. 3A is an exemplary diagram of the stomach.

FIG. 3B shows various branches of the anterior vagal trunk that innervate the stomach.

FIG. 3C shows various branches of the posterior vagal trunk that innervate the stomach.

FIG. 4 illustrates an exemplary stimulator that may be used to apply a stimulus to a stimulation site within a patient to treat obesity according to principles described herein.

FIG. 5 illustrates an exemplary microstimulator that may be used as the stimulator according to principles described herein.

FIG. 6 shows one or more catheters coupled to a microstimulator according to principles described herein.

FIG. 7 depicts a number of stimulators configured to communicate with each other and/or with one or more external devices according to principles described herein.

FIG. 8 illustrates an exemplary implanted stimulator that is coupled to the stomach according to principles described herein.

FIG. 9 illustrates an exemplary configuration wherein multiple stimulators are coupled to the stomach according to principles described herein.

FIG. 10 illustrates a stimulator that has been implanted beneath the scalp of a patient to stimulate a stimulation site within the brain associated with obesity according to principles described herein.

Throughout the drawings, identical reference numbers designate similar, but not necessarily identical, elements.

### DETAILED DESCRIPTION

The present application is related to U.S. patent application Ser. No. 11/140,152, filed May 27, 2005, which claims priority as a continuation-in-part of U.S. patent application Ser. No. 09/993,086, filed Nov. 6, 2001 and published as US2005/0033376, which claims priority based on U.S. Provisional Patent Application No. 60/252,625, filed Nov. 21, 2000. These applications are incorporated herein by reference in their respective entireties.

Methods and systems for treating obesity are described herein. An implanted stimulator is configured to apply at least one stimulus to a stimulation site within a patient in accordance with one or more stimulation parameters. The stimulus is configured to treat obesity and may include electrical stimulation, drug stimulation, gene infusion, chemical stimulation, thermal stimulation, electromagnetic stimulation, mechanical stimulation, and/or any other suitable stimulation. As used herein, and in the appended claims, “treating” obesity refers to any amelioration of one or more causes and/or one or more symptoms of obesity. For example, treating obesity as described herein may include, without being limited to, preventing weight gain, regulating gastrointestinal activity, creating a sensation of fullness such that the patient eats less, and/or reducing a sensation of hunger within the patient.

In the following description, for purposes of explanation, numerous specific details are set forth in order to provide a thorough understanding of the present systems and methods. It will be apparent, however, to one skilled in the art that the present systems and methods may be practiced without these specific details. Reference in the specification to “one embodiment” or “an embodiment” means that a particular feature, structure, or characteristic described in connection with the embodiment is included in at least one embodiment. The appearance of the phrase “in one embodiment” in various places in the specification are not necessarily all referring to the same embodiment.

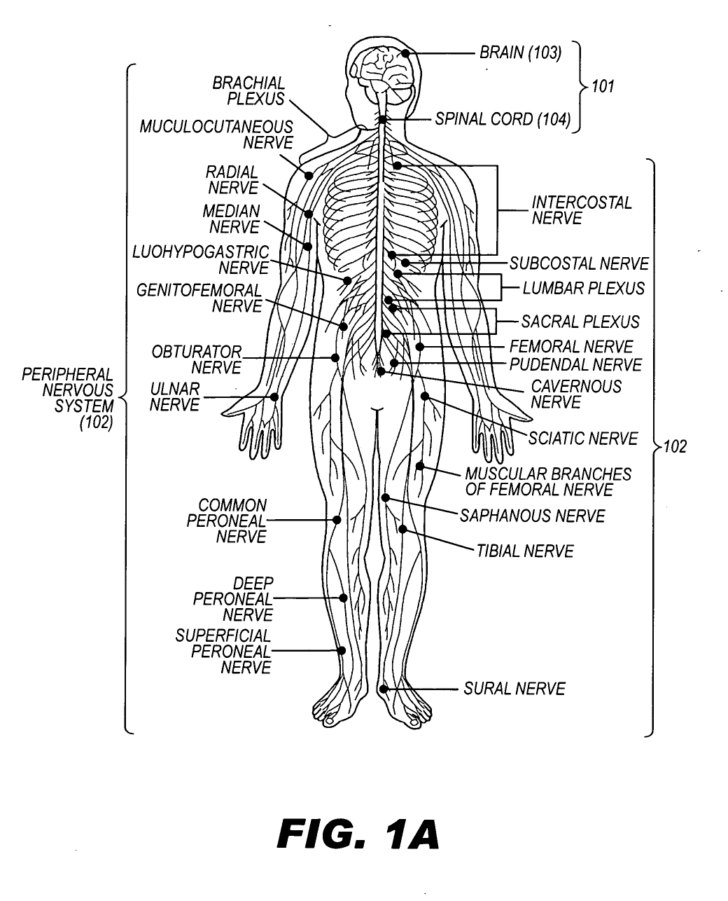

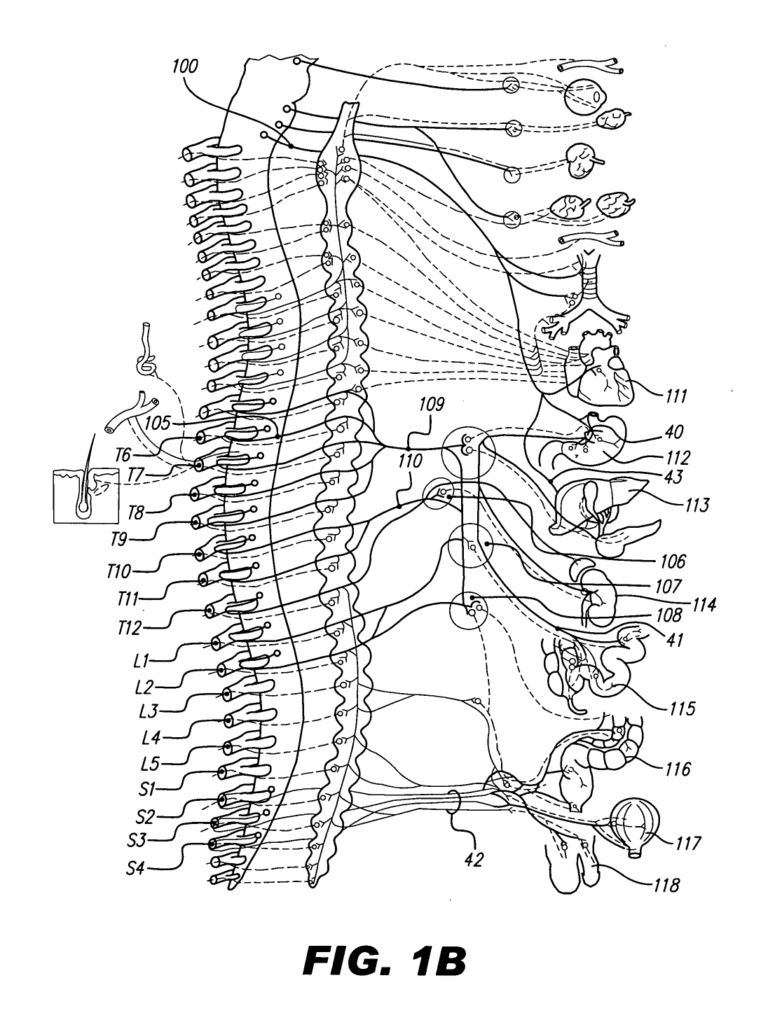

Before discussing the present methods and systems for treating obesity, a brief overview of the human nervous system, brain, and stomach will be given. FIG. 1A is a diagram of the human nervous system. The nervous system is divided into a central nervous system (101) and a peripheral nervous system (102). The central nervous system (101) includes the brain (103) and the spinal cord (104). The peripheral nervous system (102) includes a number of nerves that branch from various regions of the spinal cord (104). For example, the peripheral nervous system (102) includes, but is not limited to, the brachial plexus, the musculocutaneous nerve, the radial nerve, the median nerve, the iliohypogastric nerve, the genitofemoral nerve, the obturator nerve, the ulnar nerve, the peroneal nerve, the sural nerve, the tibial nerve, the saphenous nerve, the femoral nerve, the sciatic nerve, the cavernous nerve, the pudendal nerve, the sacral plexus, the lumbar plexus, the subcostal nerve, and the intercostal nerves.

The peripheral nervous system (102) may be further divided into the somatic nervous system and the autonomic nervous system. The somatic nervous system is the part of the peripheral nervous system (102) associated with the voluntary control of body movements through the action of skeletal muscles. The somatic nervous system consists of afferent fibers which receive information from external sources, and efferent fibers which are responsible for muscle contraction.

The autonomic nervous system, on the other hand, regulates the involuntary action of various organs and is divided into the sympathetic nervous system and the parasympathetic nervous system. FIG. 1B illustrates the autonomic nervous system. FIG. 1B shows the following structures of the parasympathetic nervous system: the anterior or posterior vagus nerves (100), the hepatic branch (43) of the vagus nerve, the celiac branch (40) of the vagus nerve, the gastric branch (41) of the vagus nerve, and branches of the pelvic plexus (42). It will be recognized that the parasympathetic nervous system also includes other structures not shown in FIG. 1B. FIG. 1B also shows the following structures of the sympathetic nervous system: the sympathetic afferent fibers (105) that exit the spinal cord at spinal levels T6, T7, T8, and T9, the sympathetic ganglia (e.g., the celiac (106) ganglia and its subsidiary plexuses, the superior mesenteric ganglia (107), and the inferior mesenteric ganglia (108), the greater splanchnic nerve (109) and the lesser splanchnic nerve (110). FIG. 1B shows a number of organs that are controlled by the autonomic nervous system, including, but not limited to, the heart (111), stomach (112), liver (113), kidney (114), large intestines (115), small intestines (116), bladder (117), and reproductive organs (118).

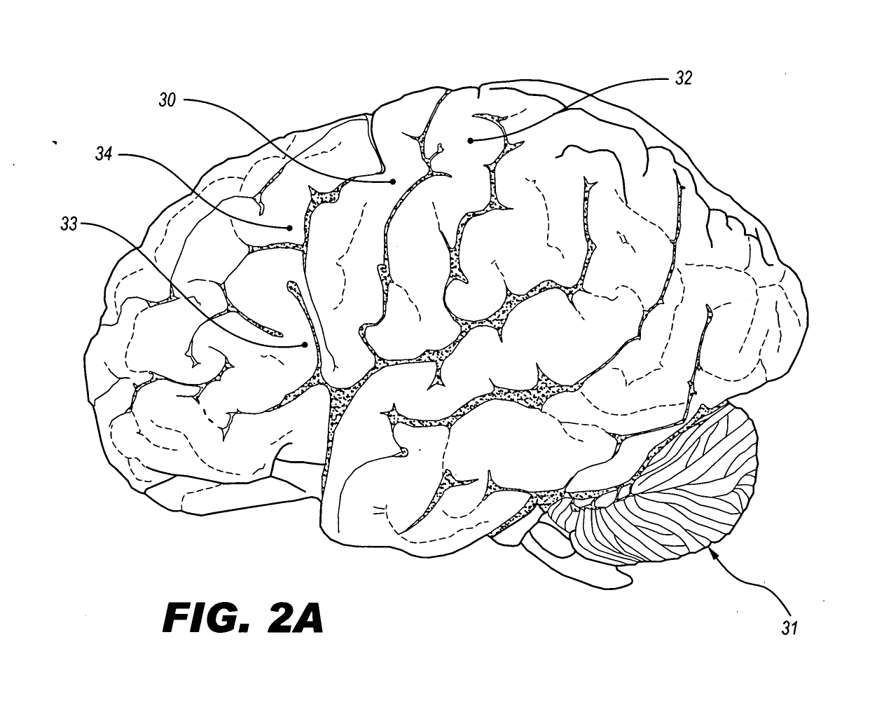

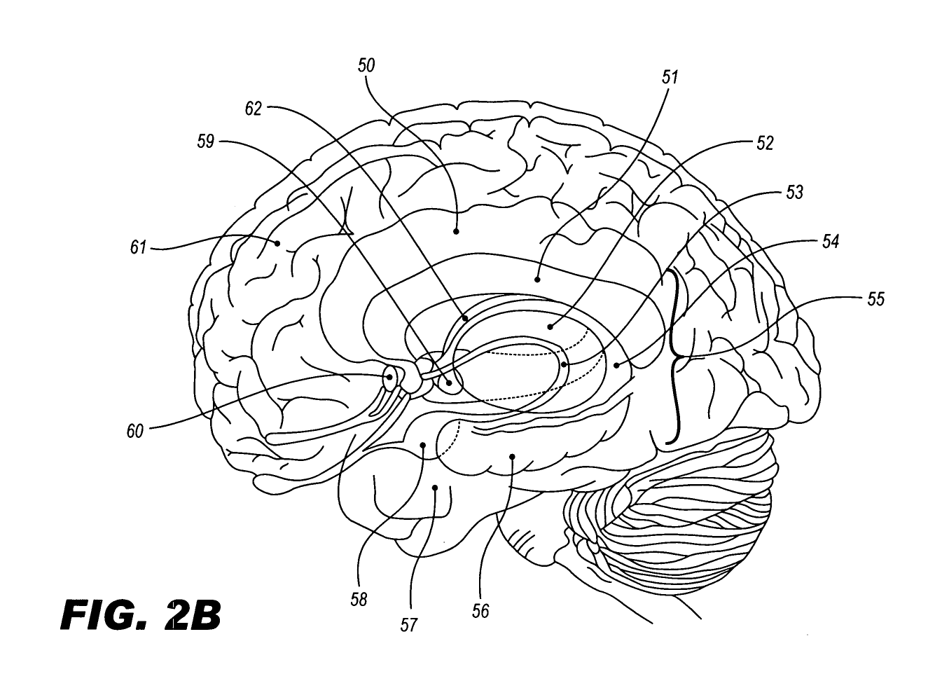

FIG. 2A depicts the lateral surface of the brain. As shown in FIG. 2A, the primary motor cortex (30) is located on the lateral surface of the brain. The primary motor cortex (30) is the cortical area that influences motor movements. Also shown in FIG. 2A are the somatosensory cortex (32), premotor cortex (33), and supplementary motor cortex (34). These structures are also involved in controlling motor movements.

FIG. 2A also shows the cerebellum (31). The cerebellum (31) is located in the posterior of the head and is responsible for the coordination of movement and balance. The cerebellum (31) includes the superior, middle and/or inferior cerebellar peduncles (not shown).

FIG. 2B depicts, in perspective view, the thalamus (52) and various structures of the brain that make up the limbic system. The thalamus (52) helps process information from the senses and relays such information to other parts of the brain. Located beneath the thalamus is the hypothalamus (not shown). The hypothalamus regulates many body functions, including appetite and body temperature.

The limbic system shown in FIG. 2B includes, but is not limited to, several subcortical structures located around the thalamus (52). Exemplary structures of the limbic system include the cingulate gyrus (50), corpus collosum (51), stria terminalis (53), caudate nucleus (54), basal ganglia (55), hippocampus (56), enterorhinal cortex (57), amygdala (58), mammillary body (59), medial septal nucleus (60), prefrontal cortex (61), and fornix (62). These structures are involved with emotion, learning, and memory.

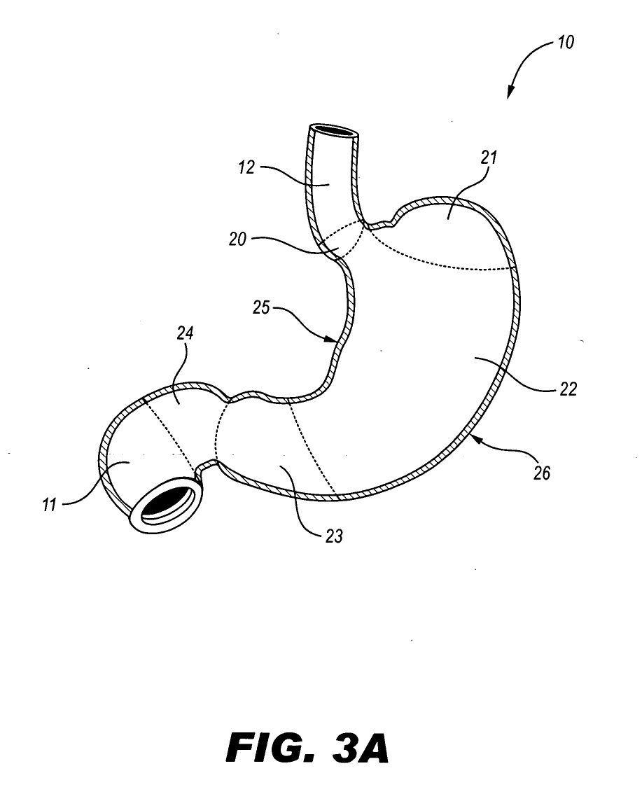

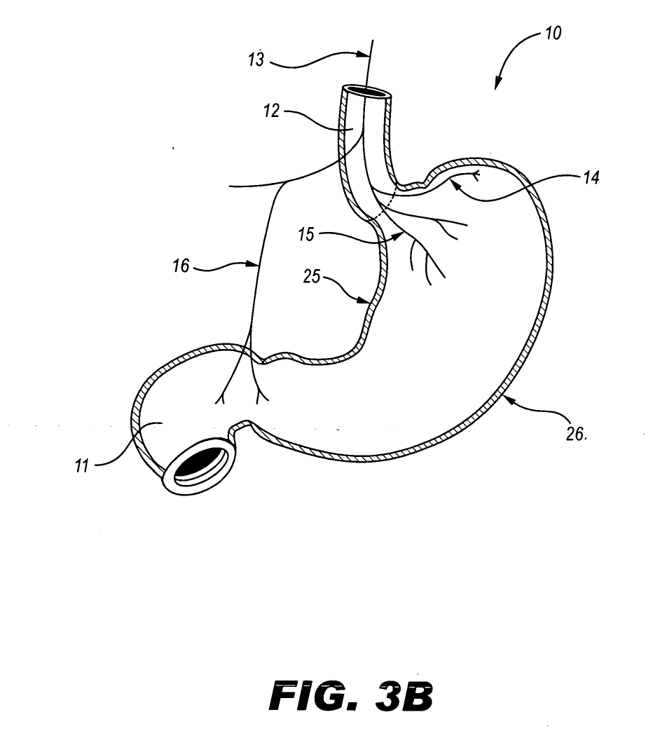

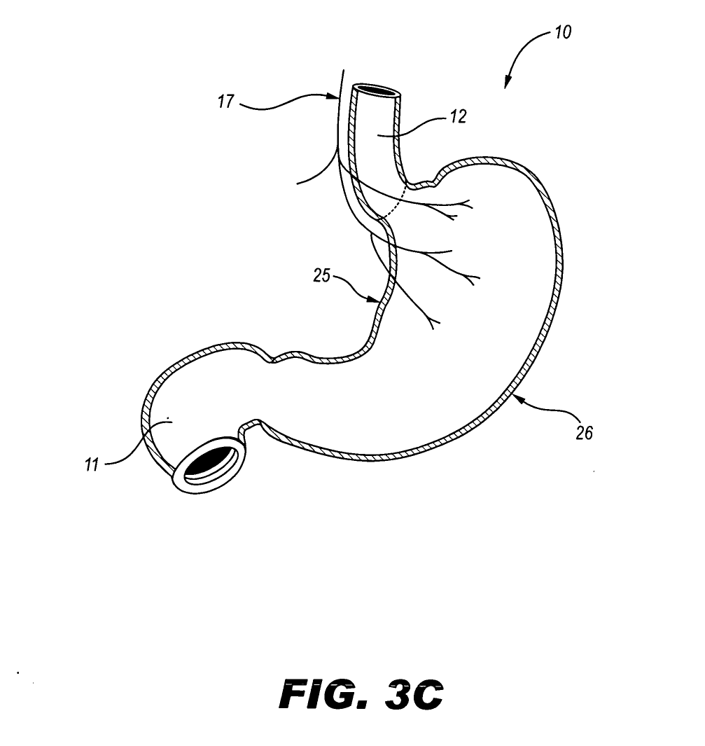

FIG. 3A is an exemplary diagram of a stomach (10). As shown in FIG. 3A, the shape of the stomach (10) as viewed laterally, has two curves: the lesser curvature (25) and the greater curvature (26), which respectively follow the upper and lower surfaces of the stomach (10). The cardia or proximal stomach (20) is located in the upper left portion of FIG. 3A and serves as the junction between the esophagus (12) and the body (22) of the stomach (10). The fundus (21), which is also located in the upper portion of the stomach (10), produces acid and pepsin that help digest food. The lower portion of the stomach (10) is known as the distal stomach and includes the antrum (23) and pylorus (24). The antrum (23) is where food is mixed with gastric juice. The pylorus (24) acts as a valve to control emptying of the stomach contents into the small intestine (11).

The stomach (10) has five nested layers of tissue. The innermost layer is where stomach acid and digestive enzymes are made and is called the mucosa. A supporting layer, know as the submucosa, surrounds the mucosa. The mucosa and submucosa are surrounded by a layer of muscle, known as the muscularis that moves and mixes the contents of the stomach. The next two layers, the subserosa and the outermost serosa, act as wrapping layers for the stomach (10).

Innervation of the stomach (10) is provided directly by the vagi nerves and through subsidiary plexuses of the celiac plexus. FIG. 3B shows various branches of the anterior vagal trunk (13), which is derived from the left vagus nerve. The hepatic branch (14) runs through the upper part of the lesser omentum and joins the plexus on the hepatic artery and portal vein. The celiac branch (15) follows the left gastric artery to the celiac plexus. The gastric branch (16), the largest of the three, follows the lesser curvature (25) of the stomach (10) and distributes anterior gastric branches to the distal portions of the stomach (10), i.e., those portions of the stomach (10) adjacent to the entrance to the small intestine (11).

FIG. 3C shows various branches of the posterior vagal trunk (17), which innervates the posterior surface of the stomach (10). The posterior vagal trunk (17) is derived largely, but not entirely from the right vagus nerve.

Both sympathetic efferent and afferent nerves to the stomach (10) are derived from T6-T9 spinal cord segments. These nerve fibers are transmitted by the greater thoracic splanchnic nerve. Preganglionic fibers relay in the celiac ganglia, and the nerves reach the stomach (10) along the branches of the celiac artery.

As used herein and in the appended claims, the term “food” will be used to refer generally to any type of nutrient-bearing substance in whatever form, e.g., solid food and/or drink that enters the stomach (10). Food that is input into the stomach (10) enters through the esophagus (12), passes through the stomach (10) and exits at the distal end of the stomach (10) into the small intestine (11). A typical stomach (10) generates electrical pulses which signal to the neurological system of a person that the stomach is full and that the person should stop eating.

The stomach (10) is emptied as a result of coordinated gastric contractions (motility). Without these coordinated contractions, digestion and absorption of dietary nutrients cannot take place. Thus, impairment of gastric contractions may result in delayed emptying of the stomach (10).

Gastric contractions are regulated by myoelectrical activity of the stomach (10), called slow waves. Gastric slow waves originate in the proximal portion of the stomach (10), e.g., near the esophagus (12), and propagate distally toward the small intestine (11). Gastric slow waves determine the maximum frequency, propagation velocity, and propagation direction of gastric contractions. The normal frequency of the gastric slow waves is about three cycles per minute (cpm) in humans. Abnormalities in gastric slow waves lead to gastric motor disorders and have been frequently observed in patients with functional disorders of the stomach, such as gastroparesis, functional dyspepsia, anorexia, etc. Some studies have shown that patients with obesity have an abnormally rapid rate of gastric slow waves.

It is believed that applying a stimulus to one or more of the locations within the body described above may be useful in treating obesity. As mentioned, “treating” obesity refers to any amelioration of one or more causes and/or one or more symptoms of obesity, such as, but not limited to, preventing weight gain, regulating gastrointestinal activity, creating a sensation of fullness such that the patient eats less, and/or reducing a sensation of hunger within a patient.

Consequently, a stimulator may be implanted in a patient to deliver a stimulus to one or more stimulation sites within the patient to treat obesity. The stimulus may include an electrical stimulation current, one or more drugs or other chemical stimulation, thermal stimulation, electromagnetic stimulation, mechanical stimulation, and/or any other suitable stimulation.

As used herein, and in the appended claims, the term “stimulator” will be used broadly to refer to any device that delivers a stimulus, such as an electrical stimulation current, one or more drugs or other chemical stimulation, thermal stimulation, electromagnetic stimulation, mechanical stimulation, and/or any other suitable stimulation at a stimulation site to treat obesity. Thus, the term “stimulator” includes, but is not limited to, a stimulator, microstimulator, implantable pulse generator (IPG), spinal cord stimulator (SCS), system control unit, cochlear implant, deep brain stimulator, drug pump, or similar device.

The stimulation site referred to herein, and in the appended claims, may include, but is not limited to, any one or more of the locations within the body described in connection with FIGS. 1A-3C. For example, the stimulation site may include, but is not limited to, a nerve in the parasympathetic nervous system, a nerve in the sympathetic nervous system, the stomach, a nerve that innervates the stomach, a blood vessel that supplies the stomach, and/or a location within the central nervous system.

Exemplary stimulation sites within the parasympathetic nervous system include, but are not limited to, the anterior vagus nerve, posterior vagus nerve, hepatic branch of the vagus nerve, celiac branch of the vagus nerve, gastric branch of the vagus nerve, and gastric nerve. Exemplary stimulation sites within the sympathetic nervous system include, but are not limited to, one or more sympathetic afferent fibers that exit the spinal cord at spinal levels T6, T7, T8, and T9; the sympathetic ganglia (e.g., superior and inferior mesenteric); the greater thoracic splanchnic nerve; the lesser thoracic splanchnic nerve; and the celiac ganglia and its subsidiary plexuses. Exemplary stimulation sites within the stomach include, but are not limited to, one or more walls of the stomach, the lesser curvature, greater curvature, cardia, fundus, antrum, pylorus, one or more layers of the stomach, and the enteric nervous system (e.g., Meissner's plexus and Auerbach's plexus). Exemplary stimulation sites within the central nervous system include, but are not limited to, the nucleus of the solitary tract, dorsal vagal complex, central nucleus of the amygdala, thalamus, hypothalamus (including lateral and ventromedial portions of the hypothalamus), spinal cord, somatosensory cortex, motor cortex, and the pleasure centers in the brain (including, but not limited to, the septum pellucidum, ventral striatum, nucleus accumbens, ventral tegmental area, limbic system, and cerebellum).

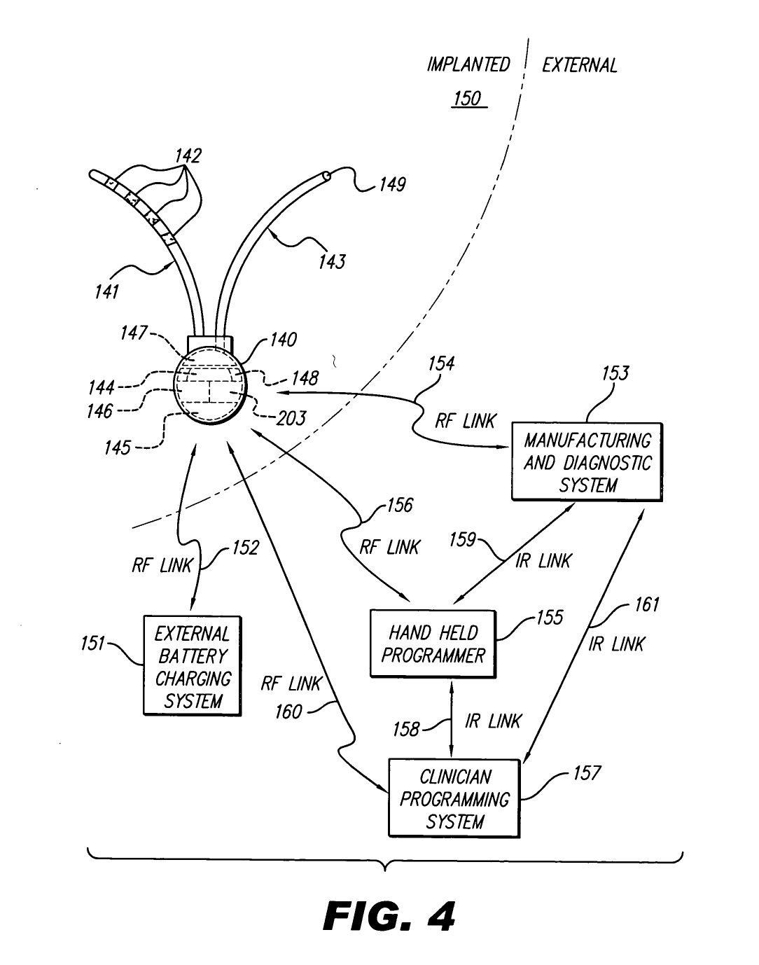

To facilitate an understanding of the methods of treating obesity with an implanted stimulator, a more detailed description of the stimulator and its operation will now be given with reference to the figures. FIG. 4 illustrates an exemplary stimulator (140) that may be implanted within a patient (150) and used to apply a stimulus to the stomach, e.g., an electrical stimulation of the stomach, an infusion of one or more drugs at the stomach, or both. The electrical stimulation function of the stimulator (140) will be described first, followed by an explanation of the possible drug delivery function of the stimulator (140). It will be understood, however, that the stimulator (140) may be configured to provide only electrical stimulation, only a drug stimulation, both types of stimulation, or any other type of stimulation as best suits a particular patient.

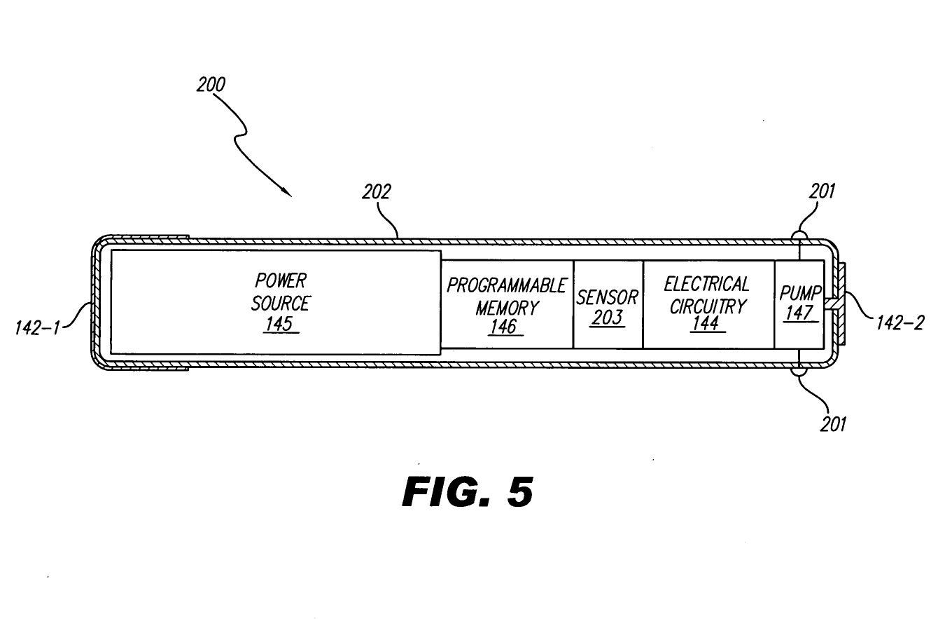

The exemplary stimulator (140) shown in FIG. 4 is configured to provide electrical stimulation to a stimulation site within a patient and may include a lead (141) having a proximal end coupled to the body of the stimulator (140). The lead (141) also includes a number of electrodes (142) configured to apply an electrical stimulation current to a stimulation site. The lead (141) may include any number of electrodes (142) as best serves a particular application. The electrodes (142) may be arranged as an array, for example, having at least two or at least four collinear electrodes. In some embodiments, the electrodes are alternatively inductively coupled to the stimulator (140). The lead (141) may be thin (e.g., less than 3 millimeters in diameter) such that the lead (141) may be positioned near a stimulation site. In some alternative examples, as will be illustrated in connection with FIG. 5, the stimulator (140) is leadless.

As illustrated in FIG. 4, the stimulator (140) includes a number of components. It will be recognized that the stimulator (140) may include additional and/or alternative components as best serves a particular application. A power source (145) is configured to output voltage used to supply the various components within the stimulator (140) with power and/or to generate the power used for electrical stimulation. The power source (145) may be a primary battery, a rechargeable battery, super capacitor, a nuclear battery, a mechanical resonator, an infrared collector (receiving, e.g., infrared energy through the skin), a thermally-powered energy source (where, e.g., memory-shaped alloys exposed to a minimal temperature difference generate power), a flexural powered energy source (where a flexible section subject to flexural forces is part of the stimulator), a bioenergy power source (where a chemical reaction provides an energy source), a fuel cell, a bioelectrical cell (where two or more electrodes use tissue-generated potentials and currents to capture energy and convert it to useable power), an osmotic pressure pump (where mechanical energy is generated due to fluid ingress), or the like. Alternatively, the stimulator (140) may include one or more components configured to receive power from another medical device that is implanted within the patient.

When the power source (145) is a battery, it may be a lithium-ion battery or other suitable type of battery. When the power source (145) is a rechargeable battery, it may be recharged from an external system through a power link such as a radio frequency (RF) power link. One type of rechargeable battery that may be used is described in International Publication WO 01/82398 A1, published Nov. 1, 2001, and/or WO 03/005465 A1, published Jan. 16, 2003, both of which are incorporated herein by reference in their respective entireties. Other battery construction techniques that may be used to make a power source (145) include those shown, e.g., in U.S. Pat. Nos. 6,280,873; 6,458,171, and U.S. Publications 2001/0046625 A1 and 2001/0053476 A1, all of which are incorporated herein by reference in their respective entireties. Recharging can be performed using an external charger.

The stimulator (140) may also include a coil (148) configured to receive and/or emit a magnetic field (also referred to as a radio frequency (RF) field) that is used to communicate with, or receive power from, one or more external devices (151, 153, 155). Such communication and/or power transfer may include, but is not limited to, transcutaneously receiving data from the external device, transmitting data to the external device, and/or receiving power used to recharge the power source (145).

For example, an external battery charging system (EBCS) (151) may provide power used to recharge the power source (145) via an RF link (152). External devices including, but not limited to, a hand held programmer (HHP) (155), clinician programming system (CPS) (157), and/or a manufacturing and diagnostic system (MDS) (153) maybe configured to activate, deactivate, program, and test the stimulator (140) via one or more RF links (154, 156). It will be recognized that the links, which are RF links (152, 154, 156) in the illustrated example, may be any type of link used to transmit data or energy, such as an optical link, a thermal link, or any other energy-coupling link. One or more of these external devices (153, 155, 157) may also be used to control the infusion of one or more drugs into the stimulation site.

Additionally, if multiple external devices are used in the treatment of a patient, there may be some communication among those external devices, as well as with the implanted stimulator (140). Again, any type of link for transmitting data or energy may be used among the various devices illustrated. For example, the CPS (157) may communicate with the HHP (155) via an infrared (IR) link (158), with the MDS (153) via an IR link (161), and/or directly with the stimulator (140) via an RF link (160). As indicated, these communication links (158, 161, 160) are not necessarily limited to IR and RF links and may include any other type of communication link. Likewise, the MDS (153) may communicate with the HHP (155) via an IR link (159) or via any other suitable communication link.

The HHP (155), MDS (153), CPS (157), and EBCS (151) are merely illustrative of the many different external devices that may be used in connection with the stimulator (140). Furthermore, it will be recognized that the functions performed by any two or more of the HHP (155), MDS (153), CPS (157), and EBCS (151) may be performed by a single external device. One or more of the external devices (153, 155, 157) may be embedded in a seat cushion, mattress cover, pillow, garment, belt, strap, pouch, or the like so as to be positioned near the implanted stimulator (140) when in use.

The stimulator (140) may also include electrical circuitry (144) configured to produce electrical stimulation pulses that are delivered to the stimulation site via the electrodes (142). In some embodiments, the stimulator (140) may be configured to produce monopolar stimulation. The stimulator (140) may alternatively or additionally be configured to produce multipolar stimulation including, but not limited to, bipolar or tripolar stimulation.

The electrical circuitry (144) may include one or more processors configured to decode stimulation parameters and generate the stimulation pulses. In some embodiments, the stimulator (140) has at least four channels and drives up to sixteen electrodes or more. The electrical circuitry (144) may include additional circuitry such as capacitors, integrated circuits, resistors, coils, and the like configured to perform a variety of functions as best serves a particular application.

The stimulator (140) may also include a programmable memory unit (146) for storing one or more sets of data and/or stimulation parameters. The stimulation parameters may include, but are not limited to, electrical stimulation parameters, drug stimulation parameters, and other types of stimulation parameters. The programmable memory (146) allows a patient, clinician, or other user of the stimulator (140) to adjust the stimulation parameters such that the stimulation applied by the stimulator (140) is safe and efficacious for treatment of a particular patient. The different types of stimulation parameters (e.g., electrical stimulation parameters and drug stimulation parameters) may be controlled independently. However, in some instances, the different types of stimulation parameters are coupled. For example, electrical stimulation may be programmed to occur only during drug stimulation or vice versa. Alternatively, the different types of stimulation may be applied at different times or with only some overlap. The programmable memory (146) may be any type of memory unit such as, but not limited to, random access memory (RAM), static RAM (SRAM), a hard drive, or the like.

The electrical stimulation parameters may control various parameters of the stimulation current applied to a stimulation site including, but not limited to, the frequency, pulse width, amplitude, waveform (e.g., square or sinusoidal), electrode configuration (i.e., anode-cathode assignment), burst pattern (e.g., burst on time and burst off time), duty cycle or burst repeat interval, ramp on time, and ramp off time of the stimulation current that is applied to the stimulation site. The drug stimulation parameters may control various parameters including, but not limited to, the amount of drugs infused at the stimulation site, the rate of drug infusion, and the frequency of drug infusion. For example, the drug stimulation parameters may cause the drug infusion rate to be intermittent, constant, or bolus. Other stimulation parameters that characterize other classes of stimuli are possible. For example, when tissue is stimulated using electromagnetic radiation, the stimulation parameters may characterize the intensity, wavelength, and timing of the electromagnetic radiation stimuli. When tissue is stimulated using mechanical stimuli, the stimulation parameters may characterize the pressure, displacement, frequency, and timing of the mechanical stimuli.

Specific stimulation parameters may have different effects on different types, causes, or symptoms of obesity and/or different patients. Thus, in some embodiments, the stimulation parameters may be adjusted by the patient, a clinician, or other user of the stimulator (140) as best serves the particular patient being treated. The stimulation parameters may also be automatically adjusted by the stimulator (140), as will be described below. For example, the stimulator (140) may increase excitement of a stimulation site by applying a stimulation current having a relatively low frequency (e.g., less than 100 Hz). The stimulator (140) may also decrease excitement of a stimulation site by applying a relatively high frequency (e.g., greater than 100 Hz). The stimulator (140) may also, or alternatively, be programmed to apply the stimulation current to a stimulation site intermittently or continuously. Different stimuli may be applied to determine which will help a particular patient feel a sensation of fullness or help the patient's stomach process food at a normal rate so as to help the patient limit the intake of unnecessary calories contributing to the obesity.

Additionally, the exemplary stimulator (140) shown in FIG. 4 is configured to provide drug stimulation to a patient by applying one or more drugs at a stimulation site within the patient. For this purpose, a pump (147) may also be included within the stimulator (140). The pump (147) is configured to store and dispense one or more drugs, for example, through a catheter (143). The catheter (143) is coupled at a proximal end to the stimulator (140) and may have an infusion outlet (149) for infusing dosages of the one or more drugs at the stimulation site. In some embodiments, the stimulator (140) may include multiple catheters (143) and/or pumps (147) for storing and infusing dosages of the one or more drugs at the stimulation site.

The pump (147) or controlled drug release device described herein may include any of a variety of different drug delivery systems. Controlled drug release devices based upon a mechanical or electromechanical infusion pump may be used. In other examples, the controlled drug release device can include a diffusion-based delivery system, e.g., erosion-based delivery systems (e.g., polymer-impregnated with drug placed within a drug-impermeable reservoir in communication with the drug delivery conduit of a catheter), electrodiffusion systems, and the like. Another example is a convective drug delivery system, e.g., systems based upon electroosmosis, vapor pressure pumps, electrolytic pumps, effervescent pumps, piezoelectric pumps and osmotic pumps. Another example is a micro-drug pump.

Exemplary pumps (147) or controlled drug release devices suitable for use as described herein include, but are not necessarily limited to, those disclosed in U.S. Pat. Nos. 3,760,984; 3,845,770; 3,916,899; 3,923,426; 3,987,790; 3,995,631; 3,916,899; 4,016,880; 4,036,228; 4,111,202; 4,111,203; 4,203,440; 4,203,442; 4,210,139; 4,327,725; 4,360,019; 4,487,603; 4,627,850; 4,692,147; 4,725,852; 4,865,845; 5,057,318; 5,059,423; 5,112,614; 5,137,727; 5,234,692; 5,234,693; 5,728,396; 6,368,315 and the like. Additional exemplary drug pumps suitable for use as described herein include, but are not necessarily limited to, those disclosed in U.S. Pat. Nos. 4,562,751; 4,678,408; 4,685,903; 5,080,653; 5,097,122; 6,740,072; and 6,770,067. Exemplary micro-drug pumps suitable for use as described herein include, but are not necessarily limited to, those disclosed in U.S. Pat. Nos. 5,234,692; 5,234,693; 5,728,396; 6,368,315; 6,666,845; and 6,620,151. All of these listed patents are incorporated herein by reference in their respective entireties.

The one or more drugs applied by the stimulator (140) may include any drug or other substance configured to treat obesity. For example, the one or more drugs that may be applied to a stimulation site to treat obesity may have an excitatory effect on the stimulation site. Additionally or alternatively, the one or more drugs may have an inhibitory effect on the stimulation site to treat obesity. Exemplary excitatory drugs that may be applied to a stimulation site to treat obesity include, but are not limited to, at least one or more of the following: an excitatory neurotransmitter (e.g., glutamate, dopamine, norepinephrine, epinephrine, acetylcholine, serotonin); an excitatory neurotransmitter agonist (e.g., glutamate receptor agonist, L-aspartic acid, N-methyl-D-aspartic acid (NMDA), bethanechol, norepinephrine); an inhibitory neurotransmitter antagonist(s) (e.g., bicuculline); an agent that increases the level of an excitatory neurotransmitter (e.g., edrophonium, Mestinon); and/or an agent that decreases the level of an inhibitory neurotransmitter (e.g., bicuculline).

Exemplary inhibitory drugs that may be applied to a stimulation site to treat obesity include, but are not limited to, at least one or more of the following: an inhibitory neurotransmitter(s) (e.g., gamma-aminobutyric acid, a.k.a. GABA, dopamine, glycine); an agonist of an inhibitory neurotransmitter (e.g., a GABA receptor agonist such as midazolam or clondine, muscimol); an excitatory neurotransmitter antagonist(s) (e.g. prazosin, metoprolol, atropine, benztropine); an agent that increases the level of an inhibitory neurotransmitter; an agent that decreases the level of an excitatory neurotransmitter (e.g., acetylcholinesterase, Group II metabotropic glutamate receptor (mGluR) agonists such as DCG-IV); a local anesthetic agent (e.g., lidocaine); and/or an analgesic medication. It will be understood that some of these drugs, such as dopamine, may act as excitatory neurotransmitters in some stimulation sites and circumstances, and as inhibitory neurotransmitters in other stimulation sites and circumstances.

Additional or alternative drugs that may be applied to a stimulation site to treat obesity include at least one or more of the following substances: one or more peptides, cholecystokinin (CCK), peptide YY (PYY), Urocortin, corticotrophin-releasing factors (CRF), sibutramine, diethylproprion, mazindol, phentermine, phenylpropanolamine, and orlistat, anesthetic agents, synthetic or natural peptides or hormones, neurotransmitters, cytokines, and other intracellular and intercellular chemicals.

Any of the drugs listed above, alone or in combination, or other drugs or combinations of drugs developed or shown to treat obesity or its symptoms may be applied to the stimulation site to treat obesity. In some embodiments, the one or more drugs are infused chronically into the stimulation site. Additionally or alternatively, the one or more drugs may be infused acutely into the stimulation site in response to a biological signal or a sensed need for the one or more drugs.

The stimulator (140) may also include a sensor device (203) configured to sense any of a number of indicators related to stomach activity, gastrointestinal activity, gastrointestinal hormone secretion, digestion, or any other factor related to obesity. For example, the sensor (203) may include a pressure sensor or transducer, a strain gauge, a force transducer, or some other device configured to sense stomach distension that occurs as a result of food intake. In some examples, the sensor (203) may be located on the lead (141). The sensor (203) may alternatively be a separate device configured to communicate with the stimulator (140). The sensor (203) will be described in more detail below.

The stimulator (140) of FIG. 4 is illustrative of many types of stimulators that may be used to apply a stimulus to a stimulation site to treat obesity. For example, the stimulator (140) may include an implantable pulse generator (IPG) coupled to one or more leads having a number of electrodes, a spinal cord stimulator (SCS), a cochlear implant, a deep brain stimulator, a drug pump (mentioned previously), a micro-drug pump (mentioned previously), or any other type of implantable stimulator configured to deliver a stimulus at a stimulation site within a patient. Exemplary IPGs suitable for use as described herein include, but are not limited to, those disclosed in U.S. Pat. Nos. 6,381,496; 6,553,263; and 6,760,626. Exemplary spinal cord stimulators suitable for use as described herein include, but are not limited to, those disclosed in U.S. Pat. Nos. 5,501,703; 6,487,446; and 6,516,227. Exemplary cochlear implants suitable for use as described herein include, but are not limited to, those disclosed in U.S. Pat. Nos. 6,219,580; 6,272,382; and 6,308,101. Exemplary deep brain stimulators suitable for use as described herein include, but are not limited to, those disclosed in U.S. Pat. Nos. 5,938,688; 6,016,449; and 6,539,263. All of these listed patents are incorporated herein by reference in their respective entireties.

Alternatively, the stimulator (140) may include an implantable microstimulator, such as a BION® microstimulator (Advanced Bionics® Corporation, Valencia, Calif.). Various details associated with the manufacture, operation, and use of implantable microstimulators are disclosed in U.S. Pat. Nos. 5,193,539; 5,193,540; 5,312,439; 6,185,452; 6,164,284; 6,208,894; and 6,051,017. All of these listed patents are incorporated herein by reference in their respective entireties.

FIG. 5 illustrates an exemplary microstimulator (200) that may be used as the stimulator (140; FIG. 4) described herein. Other configurations of the microstimulator (200) are possible, as shown in the above-referenced patents and as described further below.

As shown in FIG. 5, the microstimulator (200) may include the power source (145), the programmable memory (146), the electrical circuitry (144), and the pump (147) described in connection with FIG. 4. These components are housed within a capsule (202). The capsule (202) may be a thin, elongated cylinder or any other shape as best serves a particular application. The shape of the capsule (202) may be determined by the structure of the desired target nerve, the surrounding area, and the method of implantation. In some embodiments, the volume of the capsule (202) is substantially equal to or less than three cubic centimeters. In some embodiments, the microstimulator (200) may include two or more leadless electrodes (142) disposed on the outer surface of the microstimulator (200).

The external surfaces of the microstimulator (200) may advantageously be composed of biocompatible materials. For example, the capsule (202) may be made of glass, ceramic, metal, or any other material that provides a hermetic package that will exclude water vapor but permit passage of electromagnetic fields used to transmit data and/or power. The electrodes (142) may be made of a noble or refractory metal or compound, such as platinum, iridium, tantalum, titanium, titanium nitride, niobium or alloys of any of these, in order to avoid corrosion or electrolysis which could damage the surrounding tissues and the device.

The microstimulator (200) may also include one or more infusion outlets (201). The infusion outlets (201) facilitate the infusion of one or more drugs at a stimulation site to treat obesity. The infusion outlets (201) may dispense one or more drugs directly to the treatment site. Alternatively, catheters may be coupled to the infusion outlets (201) to deliver the drug therapy to a treatment site some distance from the body of the microstimulator (200). The stimulator (200) of FIG. 5 also includes electrodes (142-1 and 142-2) at either end of the capsule (202). One of the electrodes (142) may be designated as a stimulating electrode to be placed close to the treatment site and one of the electrodes (142) may be designated as an indifferent electrode used to complete a stimulation circuit.

The microstimulator (200) may be implanted within a patient with a surgical tool such as a hypodermic needle, bore needle, or any other tool specially designed for the purpose. Alternatively, the microstimulator (200) may be implanted using endoscopic or laparoscopic techniques.

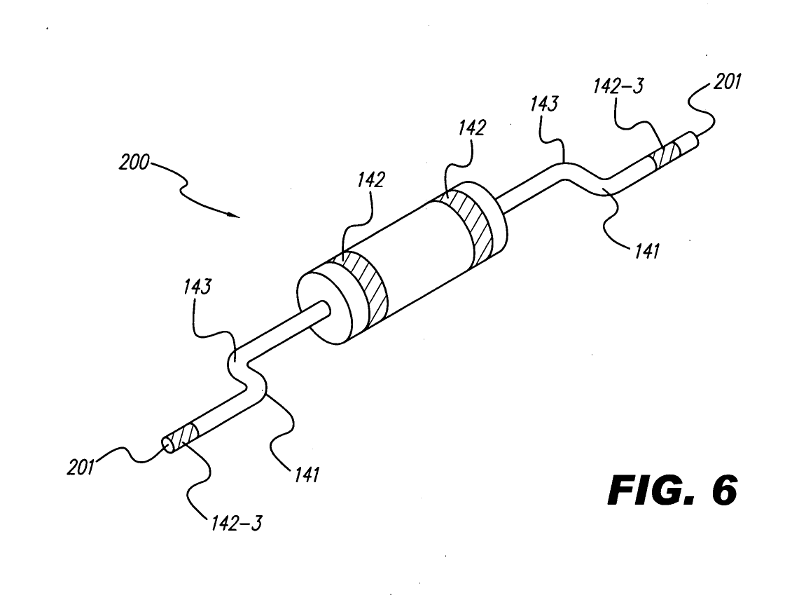

FIG. 6 shows an example of a microstimulator (200) with one or more catheters (143) coupled to the infusion outlets on the body of the microstimulator (200). With the catheters (143) in place, the infusion outlets (201) that actually deliver the drug therapy to target tissue are located at the ends of catheters (143). Thus, in the example of FIG. 6, a drug therapy is expelled by the pump (147, FIG. 5) from an infusion outlet (201, FIG. 5) in the casing (202, FIG. 5) of the microstimulator (200), through the catheter (143), out an infusion outlet (201) at the end of the catheter (143) to the stimulation site within the patient. As shown in FIG. 6, the catheters (143) may also serve as leads (141) having one or more electrodes (142-3) disposed thereon. Thus, the catheters (143) and leads (141) of FIG. 6 permit infused drugs and/or electrical stimulation current to be directed to a stimulation site while allowing most elements of the microstimulator (200) to be located in a more surgically convenient site. The example of FIG. 6 may also include leadless electrodes (142) disposed on the housing of the microstimulator (200), in the same manner described above.

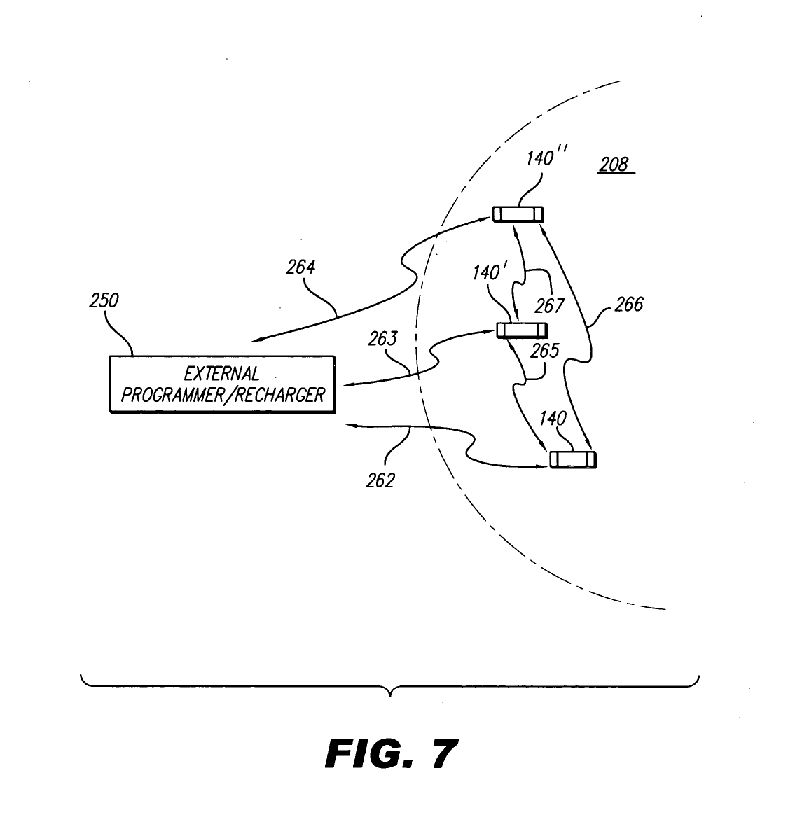

Returning to FIG. 4, the stimulator (140) may be configured to operate independently. Alternatively, as shown in FIG. 7 and described in more detail below, the stimulator (140) may be configured to operate in a coordinated manner with one or more additional stimulators, other implanted devices, or other devices external to the patient's body. For instance, a first stimulator may control, or operate under the control of, a second stimulator, other implanted device, or other device external to the patient's body. The stimulator (140) may be configured to communicate with other implanted stimulators, other implanted devices, or other devices external to the patient's body via an RF link, an untrasonic link, an optical link, or any other type of communication link. For example, the stimulator (140) may be configured to communicate with an external remote control unit that is capable of sending commands and/or data to the stimulator (140) and that is configured to receive commands and/or data from the stimulator (140).

In order to determine the strength and/or duration of electrical stimulation and/or amount and/or type(s) of stimulating drug(s) required to most effectively treat obesity, various indicators of stomach activity, obesity, and/or a patient's response to treatment may be sensed or measured. These indicators include, but are not limited to, pressure against the stomach wall, stomach distension, stomach strain, naturally occurring electrical activity within the stomach (e.g., gastric slow waves), a rate of digestion of food within the stomach, and/or any other activity within the stomach. The indicators may additionally or alternatively include gastrointestinal hormone secretion levels; electrical activity of the brain (e.g., EEG); neurotransmitter levels; hormone levels; metabolic activity in the brain; blood flow rate in the head, neck or other areas of the body; medication levels within the patient; patient input, e.g., when a patient has the urge to eat, the patient can push a button on a remote control or other external unit to initiate the stimulation; temperature of tissue in the stimulation target region; physical activity level, e.g. based on accelerometer recordings; brain hyperexcitability, e.g. increased response of given tissue to the same input; indicators of collateral tissue stimulation; and/or detection of muscle tone (mechanical strain, pressure sensor, EMG). In some embodiments, the stimulator (140) may be configured to change the stimulation parameters in a closed loop manner in response to these measurements. The sensor (203; FIG. 4) within the stimulator (140; FIG. 4) may be configured to perform the measurements. Alternatively, other sensing devices may be configured to perform the measurements and transmit the measured values to the stimulator (140).

Thus, one or more external devices may be provided to interact with the stimulator (140), and may be used to accomplish at least one or more of the following functions:

Function 1: If necessary, transmit electrical power to the stimulator (140) in order to power the stimulator (140) and/or recharge the power source (145).

Function 2: Transmit data to the stimulator (140) in order to change the stimulation parameters used by the stimulator (140).

Function 3: Receive data indicating the state of the stimulator (140) (e.g., battery level, drug level, stimulation parameters, etc.).

Additional functions may include adjusting the stimulation parameters based on information sensed by the stimulator (140) or by other sensing devices.

By way of example, an exemplary method of treating obesity may be carried out according to the following sequence of procedures. The steps listed below may be modified, reordered, and/or added to as best serves a particular application.

1. A stimulator (140) is implanted so that its electrodes (142) and/or infusion outlet (149) are in communication with a stimulation site (e.g., the stomach). As used herein and in the appended claims, the term “in communication with” refers to the stimulator (140), stimulating electrodes (142), and/or infusion outlet (149) being adjacent to, in the general vicinity of, in close proximity to, directly next to, or directly on the stimulation site.

2. The stimulator (140) is programmed to apply at least one stimulus to the stimulation site. The stimulus may include electrical stimulation, drug stimulation, gene infusion, chemical stimulation, thermal stimulation, electromagnetic stimulation, mechanical stimulation, and/or any other suitable stimulation.

3. When the patient desires to invoke stimulation, the patient sends a command to the stimulator (140) (e.g., via a remote control) such that the stimulator (140) delivers the prescribed stimulation. The stimulator (140) may be alternatively or additionally configured to automatically apply the stimulation in response to sensed indicators of obesity.

4. To cease stimulation, the patient may turn off the stimulator (140) (e.g., via a remote control).

5. Periodically, the power source (145) of the stimulator (140) is recharged, if necessary, in accordance with Function 1 described above. As will be described below, this recharging function can be made much more efficient using the principles disclosed herein.

In other examples, the treatment administered by the stimulator (140), i.e., drug therapy and/or electrical stimulation, may be automatic and not controlled or invoked by the patient.

For the treatment of different patients, it may be desirable to modify or adjust the algorithmic functions performed by the implanted and/or external components, as well as the surgical approaches. For example, in some situations, it may be desirable to employ more than one stimulator (140), each of which could be separately controlled by means of a digital address. Multiple channels and/or multiple patterns of stimulation may thereby be used to deal with various symptoms or causes of obesity or various combinations of medical conditions.

As shown in the example of FIG. 7, a first stimulator (140) implanted within the patient (208) provides a stimulus to a first location; a second stimulator (140′) provides a stimulus to a second location; and a third stimulator (140″) provides a stimulus to a third location. As mentioned earlier, the implanted devices may operate independently or may operate in a coordinated manner with other implanted devices or other devices external to the patient's body. That is, an external controller (250) may be configured to control the operation of each of the implanted devices (140, 140′, and 140″). In some embodiments, an implanted device, e.g. stimulator (140), may control, or operate under the control of, another implanted device(s), e.g. stimulator (140′) and/or stimulator (140″). Control lines (262-267) have been drawn in FIG. 7 to illustrate that the external controller (250) may communicate or provide power to any of the implanted devices (140, 140′, and 140″) and that each of the various implanted devices (140, 140′, and 140″) may communicate with and, in some instances, control any of the other implanted devices.

As a further example of multiple stimulators (140) operating in a coordinated manner, the first and second stimulators (140, 140′) of FIG. 7 may be configured to sense various indicators of the symptoms or causes of obesity and transmit the measured information to the third stimulator (140″). The third stimulator (140″) may then use the measured information to adjust its stimulation parameters and apply stimulation to a stimulation site accordingly. The various implanted stimulators may, in any combination, sense indicators of obesity, communicate or receive data on such indicators, and adjust stimulation parameters accordingly.

Alternatively, the external device (250) or other external devices communicating with the external device may be configured to sense various indicators of a patient's condition. The sensed indicators can then be collected by the external device (250) for relay to one or more of the implanted stimulators or may be transmitted directly to one or more of the implanted stimulators by any of an array of external sensing devices. In either case, the stimulator, upon receiving the sensed indicator(s), may adjust stimulation parameters accordingly. In other examples, the external controller (250) may determine whether any change to stimulation parameters is needed based on the sensed indicators. The external device (250) may then signal a command to one or more of the stimulators to adjust stimulation parameters accordingly.

The stimulator (140) of FIG. 4 may be implanted within a patient using any suitable surgical procedure such as, but not limited to, injection, small incision, open placement, laparoscopy, or endoscopy. Exemplary methods of implanting a microstimulator, for example, are described in U.S. Pat. Nos. 5,193,539; 5,193,540; 5,312,439; 6,185,452; 6,164,284; 6,208,894; and 6,051,017. Exemplary methods of implanting an SCS, for example, are described in U.S. Pat. Nos. 5,501,703; 6,487,446; and 6,516,227. Exemplary methods of implanting a deep brain stimulator, for example, are described in U.S. Pat. Nos. 5,938,688; 6,016,449; and 6,539,263. All of these listed patents are incorporated herein by reference in their respective entireties.

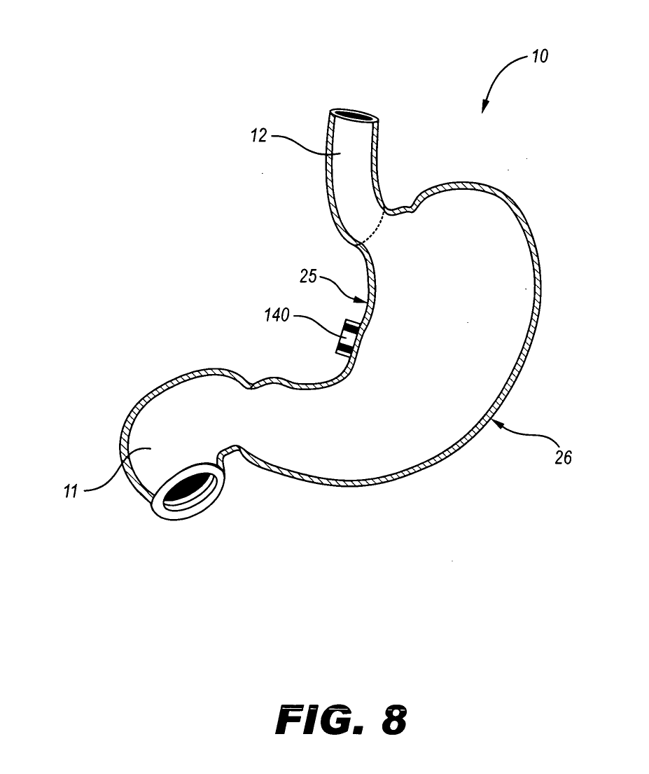

By way of example, FIG. 8 illustrates an exemplary implanted stimulator (140) that is coupled to the stomach (10) to provide stimulation to the stomach (10). The stimulator is coupled to the lesser curvature (25) of the stomach (10) in FIG. 8 for illustrative purposes only. It will be recognized that the stimulator (140) may be coupled to any portion of the stomach (10) as best serves a particular application. For example, the stimulator (140) may be coupled to the greater curvature (26), cardia (20; FIG. 1A), fundus (21; FIG. 1A), antrum (23; FIG. 1A), pylorus (24; FIG. 1A), or any portion of the body (22; FIG. 1A) of the stomach (10). Additionally or alternatively, the stimulator (140) may be coupled to any of the five layers of the stomach (10), to a nerve that innervates the stomach (10), or to a blood vessel that supplies the stomach (10).

The stimulator (140) may be secured to the stomach (10) or to any other location within the body using any of a number of techniques. In some examples, the stimulator (140) is sutured to the stomach (10) using one or more sutures. Alternatively, a medical adhesive, insulative backing, hook, barb, or other securing device or material may be used to secure the stimulator (140) at a desired location.

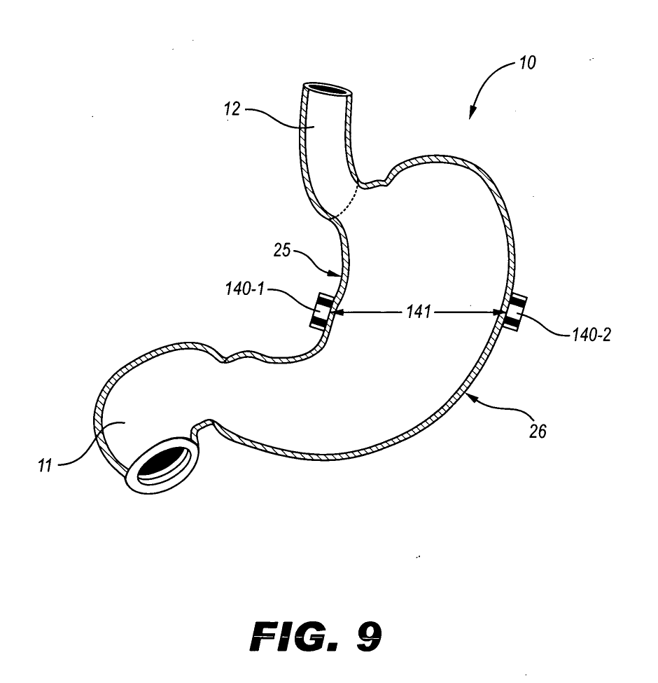

As mentioned, multiple stimulators (140) may be implanted within a patient and configured to operate in a coordinated manner to treat obesity. For example, FIG. 9 illustrates an exemplary configuration wherein multiple stimulators (140-1, 140-2) are coupled to the stomach (10) to provide stimulation to the stomach (10). FIG. 9 shows a first stimulator (140-1) coupled to the lesser curvature (25) of the stomach (10) and a second stimulator (140-1) coupled to the greater curvature (26) of the stomach (10). However, it will be recognized that any number of stimulators (140) may be coupled to any portion of the stomach (10) as best serves a particular application.

In some examples, stomach distension may be sensed by measuring the distance between two stimulators (140) that are coupled to the stomach (10). For example, the separation distance (141) between the stimulators (140-1, 140-2) of FIG. 9 may be measured to sense stomach distension. As the stomach (10) distends due to the intake of food, the separation distance (141) increases. One or more of the stimulators (140-1, 140-2) may then turn on, adjust, or turn off stimulation to the stomach (10) in response to this change in separation distance (141) between the stimulators (140-1, 140-2).

In some embodiments, the stimulators (140-1, 140-2) are configured to sense the separation distance (141) by communicating with each other using one or more RF fields. For example, the first stimulator (140-1) may be configured to transmit an RF field and the second stimulator (140-2) may be configured to sense the signal strength of the RF field transmitted by the first stimulator (140-1). When the stomach (10) distends due to an intake of food, the separation distance (141) between the two stimulators (140-1, 140-2) increases, thereby decreasing the sensed signal strength of the transmitted RF field. The second stimulator (140-2) senses this decrease in signal strength of the RF field transmitted by the first stimulator (140-1). One or more of the stimulators (140-1, 140-2) may then stimulate the stomach (10) in response to this decrease in sensed signal strength of the transmitted RF field. The stimulation applied may be proportional to the decrease in signal strength of the transmitted RF field allowing for a continuum of possible stimulation levels dictated by the amount of stomach distention. It will be recognized that the first and second stimulators (140-1, 140-2) may communicate via any suitable communication link including, but not limited to, an infrared (IR) link, an optical link, or Bluetooth™.

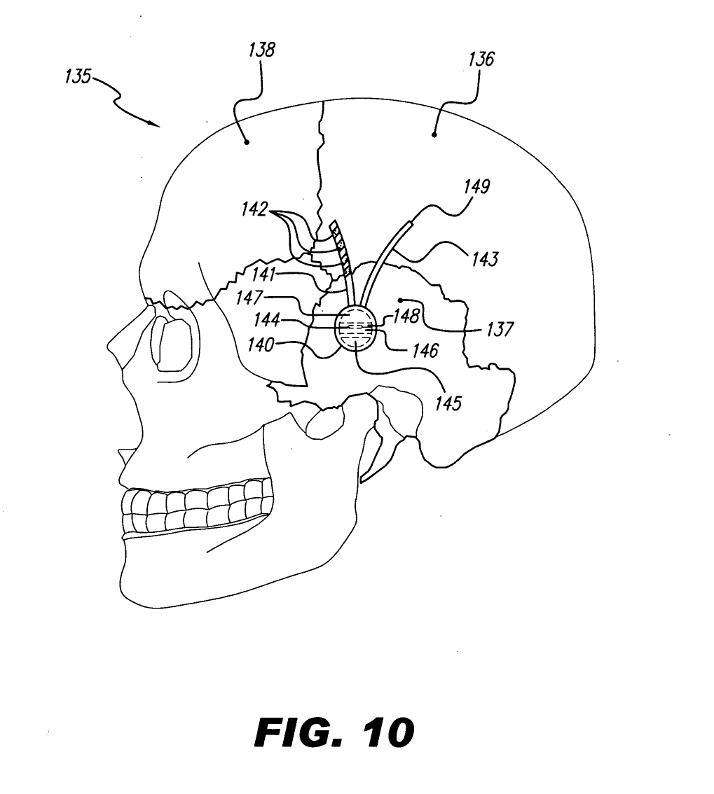

The stimulator (140) may alternatively be implanted beneath the scalp of a patient to stimulate a stimulation site within the brain. For example, as shown in FIG. 10, the stimulator (140) may be implanted in a surgically-created shallow depression or opening in the skull (135). The depression maybe made in the parietal bone (136), temporal bone (137), frontal bone (138), or any other bone within the skull (135) as best serves a particular application. The stimulator (140) may conform to the profile of surrounding tissue(s) and/or bone(s), thereby minimizing the pressure applied to the skin or scalp. Additionally or alternatively, the stimulator (140) may be implanted in a subdural space over any of the lobes of the brain, in a sinus cavity, or in an intracerebral ventricle.

In some embodiments, as shown in FIG. 10, a lead (141) and/or catheter (143) run subcutaneously to an opening in the skull (135) and pass through the opening such that it is in communication with a stimulation site in the brain. Alternatively, the stimulator (140) is leadless and is configured to generate a stimulus that passes through the skull. In this manner, a stimulation site within the brain may be stimulated without having to physically invade the brain itself.

It will be recognized that the implant locations of the stimulator (140) illustrated in FIGS. 8-10 are merely illustrative and that the stimulator (140) may additionally or alternatively be implanted in any other suitable location within the body.

In some examples, the stimulator (140) enables or turns on the stimulation at a stimulation site when the sensor (203; FIG. 4) senses one or more indicators of stomach activity, digestion, or other factors related to obesity. For example, the stimulator (140) may be configured to enable stimulation of a stimulation site when the sensor (203; FIG. 4) senses stomach distension or electrical activity produced by the stomach.

The various stimulation parameters (e.g., frequency, pulse width, amplitude, electrode polarity configuration, burst pattern, duty cycle, ramp on time, ramp off time, drug quantity, drug infusion rate, and drug infusion frequency) associated with the stimulation may be continuously adjusted in response to the sensed obesity factors. In some examples, the stimulation parameters are automatically adjusted by the stimulator (140) in response to the sensed obesity factors. For example, the stimulator (140) may automatically increase the frequency and/or amplitude of the stimulation if the sensor (203; FIG. 4) senses an increase in stomach distension or electrical activity produced by the stomach. The stimulation causes the patient to feel a sensation of fullness before the stomach fully distends such that the patient eats less. The stimulation may additionally or alternatively reduce a sensation of hunger such that the patient eats less.

The stimulator (140) may additionally or alternatively be configured to stimulate a stimulation site during periods of time in which the patient is not eating so that the patient feels a sensation of fullness, thereby reducing the patient's desire to eat. The frequency of stimulation may be programmed and adjusted as best serves a particular patient.

In some examples, the stimulator (140) is configured to provide intermittent stimulation to a stimulation site. Intermittent stimulation is also referred to as demand pacing stimulation. In intermittent stimulation, the stimulator (140) is configured to intermittently disable or turn off the stimulation to a stimulation site. Intermittent stimulation increases the effectiveness of the stimulation for some obese patients by preventing the stimulation site from adapting to the stimulation. Intermittent stimulation is also beneficial in many applications because it requires less battery power than does continuous stimulation. Hence, the stimulator (140) may operate longer without being recharged, the power source (145; FIG. 4) may be smaller, and the overall size of the stimulator (140) may be reduced.

In some examples, the stimulation applied by the stimulator (140) may be configured to treat obesity by causing one or more sections of stomach to remain in a contracted state. With these sections contracted, other sections of the stomach stretch more than they normally would when food enters the stomach, thereby creating the sensation of fullness and causing the patient to eat less.

In some alternative examples, stimulation of a stimulation site (e.g., one or more areas within the central nervous system) may be configured to mask or reduce the perception of hunger experienced by the patient. In this manner, the patient will be less likely to overeat.

The preceding description has been presented only to illustrate and describe embodiments of the invention. It is not intended to be exhaustive or to limit the invention to any precise form disclosed. Many modifications and variations are possible in light of the above teaching.

What is claimed is:

1. A method of treating obesity, said method comprising:
applying at least one stimulus with an implanted stimulator to a stimulation site within a patient;
wherein said stimulus is in accordance with one or more stimulation parameters and configured to treat said obesity.

2. The method of claim 1, wherein said stimulation site comprises at least one or more locations in communication with a parasympathetic nervous system, a sympathetic nervous system, a stomach, and a central nervous system of said patient.

3. The method of claim 1, wherein said stimulation site comprises at least one or more of a blood vessel that supplies a stomach of said patient, an anterior vagus nerve, a posterior vagus nerve, a hepatic branch of said vagus nerve, a celiac branch of said vagus nerve, gastric branch of said vagus nerve, a gastric nerve, a sympathetic afferent fiber, a sympathetic ganglia, a greater thoracic splanchnic nerve, a lesser thoracic splanchnic nerve, a celiac ganglia, one or more walls of said stomach, a lesser curvature of said stomach, a greater curvature of said stomach, a cardia of said stomach, a fundus of said stomach, an antrum of said stomach, a pylorus of said stomach, a layer of said stomach, a nerve in an enteric nervous system, a nucleus of a solitary tract, a dorsal vagal complex, an amygdala, a thalamus, a hypothalamus, a spinal cord, a somatosensory cortex, a motor cortex, a septum pellucidum, a ventral striatum, a nucleus accumbens, a ventral tegmental area, a structure in a limbic system, and a cerebellum.

4. The method of claim 1, wherein said stimulator is coupled to one or more electrodes, and wherein said stimulus comprises a stimulation current delivered via said electrodes.

5. The method of claim 1, wherein said stimulus comprises one or more drugs delivered to said stimulation site.

6. The method of claim 1, wherein said stimulus comprises a stimulation current delivered to said stimulation site and one or more drugs delivered to said stimulation site.

7. The method of claim 1, wherein said stimulus is configured to create a sensation of fullness within said patient.

8. The method of claim 1, wherein said stimulus is configured to regulate gastrointestinal activity within said patient.

9. The method of claim 1, further comprising sensing at least one physical parameter of said patient related to obesity using said at least one sensed indicator to adjust one or more of said stimulation parameters.

10. The method of claim 9, wherein said one or more physical parameters comprise at least one or more of a distension of said stomach, a strain of said stomach, an electrical signal produced by said stomach, a rate of digestion of food within said stomach, food intake into said stomach, one or more gastric slow waves produced by said stomach, an electrical activity in the brain of said patient, a gastrointestinal hormone secretion level, a neurotransmitter level, a hormone level, a metabolic activity, a blood flow rate, a medication level, a temperature of tissue in said patient, and a physical activity level of said patient.

11. A system for treating obesity, said system comprising:
a stimulator configured to apply at least one stimulus to a stimulation site within a patient in accordance with one or more stimulation parameters;
wherein said stimulation parameters and resulting stimulus are configured to treat said obesity.

12. The system of claim 11, wherein said stimulation site comprises at least one or more locations in communication with a parasympathetic nervous system, a sympathetic nervous system, a stomach, and a central nervous system of said patient.

13. The system of claim 11, wherein said stimulation site comprises at least one or more of a blood vessel that supplies a stomach of said patient, an anterior vagus nerve, a posterior vagus nerve, a hepatic branch of said vagus nerve, a celiac branch of said vagus nerve, gastric branch of said vagus nerve, a gastric nerve, a sympathetic afferent fiber, a sympathetic ganglia, a greater thoracic splanchnic nerve, a lesser thoracic splanchnic nerve, a celiac ganglia, one or more walls of said stomach, a lesser curvature of said stomach, a greater curvature of said stomach, a cardia of said stomach, a fundus of said stomach, an antrum of said stomach, a pylorus of said stomach, a layer of said stomach, a nerve in an enteric nervous system, a nucleus of a solitary tract, a dorsal vagal complex, an amygdala, a thalamus, a hypothalamus, a spinal cord, a somatosensory cortex, a motor cortex, a septum pellucidum, a ventral striatum, a nucleus accumbens, a ventral tegmental area, a structure in a limbic system, and a cerebellum.

14. The system of claim 11, wherein said stimulator is coupled to one or more electrodes, and wherein said stimulus comprises a stimulation current delivered via said electrodes.

15. The system of claim 11, wherein said stimulator comprises a drug delivery system and said stimulus comprises one or more drugs delivered to said stimulation site via said drug delivery system.

16. The system of claim 11, wherein said stimulus comprises a stimulation current delivered to said stimulation site and one or more drugs delivered to said stimulation site.

17. The system of claim 11, further comprising:
one or more sensor devices configured to sense one or more physical parameters of said patient related to obesity;
wherein said stimulator uses said one or more sensed physical parameters to adjust one or more of said stimulation parameters.

18. The system of claim 17, wherein said one or more physical parameters comprise at least one or more of a distension of said stomach, a strain of said stomach, an electrical signal produced by said stomach, a rate of digestion of food within said stomach, food intake into said stomach, one or more gastric slow waves produced by said stomach, an electrical activity in the brain of said patient, a gastrointestinal hormone secretion level, a neurotransmitter level, a hormone level, a metabolic activity, a blood flow rate, a medication level, a temperature of tissue in said patient, and a physical activity level of said patient.

19. A system for treating obesity, said system comprising:
means for applying at least one stimulus to a stimulation site within a patient in accordance with one or more stimulation parameters; and
means for adjusting said stimulation parameters such that said stimulus is effective to treat obesity.

20. The system of claim 19, wherein said stimulation site comprises at least one or more locations in communication with a parasympathetic nervous system, a sympathetic nervous system, a stomach, and a central nervous system of said patient.
